(19) United States
(12) Patent Application Publication
(10) Pub. No.: US 2024/0189464 A1
(43) Pub. Date: Jun. 13, 2024

(54) Disinfecting Light Emitting Subcomponent

(71) Applicant: Vyv, Inc., Latham, NY (US)

(72) Inventors: Robert Barron, Boulder, CO (US); Cori Winslow, Rensselaer, NY (US); Jorel Lalicki, Troy, NY (US); James Peterson, Falls Church, VA (US)

(21) Appl. No.: 18/544,661

(22) Filed: Dec. 19, 2023

Related U.S. Application Data
(63) Continuation of application No. 16/577,766, filed on Sep. 20, 2019, now Pat. No. 11,878,084.

(51) Int. Cl. A61L 2/08; A61L 2/24

(52) U.S. Cl. CPC A61L 2/084; A61L 2/24; A61L 2202/11; A61L 2202/14

(57) ABSTRACT

Methods and systems involving disinfecting light subcomponents are provided. An example system may include a substrate with one or more light emitters disposed on the substrate. The one or more light emitters may be configured to inactivate microorganisms on a surface by emitting light. The light may comprise a proportion of spectral energy of the light, measured in a 380 nanometers (nm) to 420 nm wavelength range. The light may comprise an irradiance at the surface sufficient to initiate inactivation of microorganisms on the surface.

### CROSS REFERENCE TO RELATED APPLICATIONS

This application claims the benefit of U.S. patent application Ser. No. 16/577,766, filed on Sep. 20, 2023. The above-referenced application is hereby incorporated by reference in its entirety.

### TECHNICAL FIELD

Aspects of the present disclosure generally relate to processes, systems, and apparatus for disinfecting light emitting subcomponents.

### BACKGROUND

Many surfaces may be inhabited by harmful microorganisms: bacteria, mold, fungi, etc. due to the nature of their function. For example, a cleaning device (e.g., a mop) may comprise bacteria on its cleaning surfaces. As another example, surfaces such as a countertop used for food preparation may comprise bacteria due to human interaction. Microorganisms may transfer through contact with a surface, e.g., touching a door handle, and may cause illness to the users. Surfaces inhabited by harmful microorganisms may be external, e.g., countertops, or internal to a device, e.g., inside a humidifier. Harmful bacteria such as Escherichia coli (E. coli), Salmonella, Methicillin-resistant Staphylococcus aureus (MRSA), and Clostridium difficile may be found on many surfaces, and may increase the chance of a user becoming sick or transmitting the bacteria. For example, many surfaces within a kitchen, e.g., cutting boards, may come into contact with raw meat and vegetables which may contain bacteria that may lead to food-borne illnesses. Microorganisms located on internal or external surfaces may create unpleasant odors, e.g., bacteria on a wet mop, or create unpleasant visible effects, e.g., mold on shower surfaces. Harmful microorganisms may also be harbored on surfaces in commercial settings, such as, for example, food production lines, indoor agriculture grow rooms, and healthcare settings.

### SUMMARY

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary merely presents some concepts of the disclosure in a simplified form as a prelude to the description below.

In some examples, a light emitting device may comprise a substrate and one or more light emitters disposed on the substrate. The light emitters may be configured to inactivate microorganisms on a surface a distance away from the substrate by emitting a light. The light may comprise a proportion of a spectral energy of the light, measured in a 380 nanometers (nm) to 420 nm wavelength range, greater than 50%. The light may comprise a full width half max (FWHM) emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface.

In some examples, a method may comprise emitting, via one or more light emitters disposed on a substrate and to inactivate microorganisms on a surface a distance away from the substrate, a light. The light may comprise a proportion of a spectral energy of the light, measured in a 380 nm to 420 nm wavelength range, greater than 50%. The light may comprise a FWHM emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface. The light may cause, based on emission of the light, inactivation of the microorganisms on the surface.

In some examples, a light emitting device may comprise a substrate and an array of light emitting subcomponents disposed on the substrate. The light emitting subcomponents may be configured to inactivate microorganisms on a surface a distance away from the substrate by emitting a light. The light may comprise a proportion of a spectral energy of the light, measured in a 380 nanometers (nm) to 420 nm wavelength range, greater than 50%. The light may comprise a FWHM emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface. The light emitting device may comprise a controller disposed on the substrate and configured to adjust output of the array of light emitting subcomponents.

### BRIEF DESCRIPTION OF THE DRAWINGS

Examples herein will be described in detail, with reference to the following figures, wherein like designations denote like elements.

FIGS. 1A-1B illustrate different views of an example light emitter on a substrate.

FIGS. 1C-1D illustrate different views of another example light emitter on a substrate.

FIGS. 2A-2B illustrate different views of an example light emitter, on a substrate, with two lighting elements.

FIGS. 2C-2D illustrate different views of another example light emitter, on a substrate, with two lighting elements.

FIGS. 3A-3B illustrate different views of an example light emitter, on a substrate, with four lighting elements.

FIGS. 4A-4B illustrate example strip light emitting subcomponent.

FIGS. 5A-5B illustrate example circular light emitting subcomponent.

FIG. 6 illustrates an example rectangular light emitting subcomponent.

FIGS. 7A-7B illustrate an example relationship between LED beam angles and a corresponding illuminated surface to cause inactivation.

FIG. 8 illustrates an example light emitter and a corresponding substrate and housing.

### DETAILED DESCRIPTION

In the following description of the various examples, reference is made to the accompanying drawings, which form a part hereof, and in which is shown by way of illustration, various examples of the disclosure that may be practiced. It is to be understood that other examples may be utilized.

Surfaces may be disinfected in a number of ways. One technique may be cleaning with disinfecting chemical cleaners or soaps. Chemical cleaners may only provide intermittent disinfection, which may allow harmful microorganisms to build up between cleanings. Some disinfecting systems transmit ultraviolet (UV) light onto surfaces for disinfection. UV light exposure may be harmful for humans and animals, so UV light should be off when there may be a chance of user exposure. Accordingly, these systems may involve complex controls to prevent harmful, direct exposure to humans. Additionally, UV light may cause degradation and yellowing of material (e.g., plastic) and/or surfaces.

Wavelengths of visible light in the violet range, 380-420 nanometer (nm) (e.g., 405 nm), may have a lethal effect on microorganisms such as bacteria, yeast, mold, and fungi. For example, Escherichia coli (E. coli), Salmonella, Methicillin-resistant Staphylococcus aureus (MRSA), and Clostridium difficile may be susceptible to 380-420 nm light. These wavelengths may initiate a photoreaction with porphyrin molecules found in such microorganisms. The porphyrin molecules may be photoactivated and may react with other cellular components to produce Reactive Oxygen Species (ROS). ROS may cause irreparable cell damage and eventually destroy, kill, or otherwise inactivate cells of microorganisms. Because humans, plants, and/or animals do not contain the same porphyrin molecules, this technique may be completely safe for human exposure.

In some examples, inactivation, in relation to microorganism death, may include control of and/or reduction in microorganism colonies or individual cells when exposed to disinfecting light for a certain duration.

In some examples, light emitting subcomponents may be configured to disinfect by providing continuous and/or longer-term intermittent disinfection through the use of safe visible light. In some examples, longer-term intermittent disinfection may comprise continuous disinfection with minimal interruptions, e.g., disinfection for days or weeks without interruption. In some examples, longer-term intermittent disinfection may comprise continuous disinfection for at least 2 hours. In some examples, longer-term intermittent disinfection may comprise continuous disinfection for approximately 8 hours, e.g., overnight. The example light emitting subcomponents disclosed herein may be configured to be integrated into a larger assembly, e.g., a fixture or device.

Examples disclosed herein may include a subcomponent comprising at least one light emitter, wherein the at least one light emitter may be configured to emit light having a wavelength in the range of 380-420 nm. Light emitters may comprise any device capable of emitting light, such as, for example, semiconductor die, LEDs, LEDs with light-converting layer(s), laser, electroluminescent wires, electroluminescent sheets, flexible LEDs, or OLEDs. Light emitters, as described herein, may comprise any now known or later developed material appropriate for the stated wavelength such as, for example, indium gallium nitride (InGaN) or gallium nitride (GaN).

As disclosed herein, light with a peak wavelength of light, or in some examples, multiple peak wavelengths, in a range of approximately 380 nm 420 nm may be utilized for the inactivation of bacterial pathogens. For example, approximately 405 nm light may be used as the peak wavelength. In some examples, any wavelength within 380 nm to 420 nm may be utilized, and that the peak wavelength may include a specific wavelength plus or minus approximately 5 nm.

Radiant flux, measured in Watts, is the total power from a light source. Irradiance is the power per unit area at a distance away from the light source. In some examples, the target irradiance on a target surface from the light source may be 10 mW/cm². A 10mW/cm² target irradiance may be provided, for example, by a light source with a radiant flux of 10 mW located 1 cm from the target surface. In another example, a light source may be located 5 cm from the target surface. With a target irradiance of 10 mW/cm², the light source may be configured to produce a radiant flux approximately 250 mW. These calculations may be approximately based on the inverse square law, as shown in Equation 1, where the excitation light source may be assumed to be a point source, E is the irradiance, I is the radiant flux, and r is the distance from the excitation light source to a target surface.

$$E ≅ \frac{I}{{r}^{2}} \qquad Equation 1$$

A minimum irradiance of light (e.g., in the 380-420 nm wavelength) on a surface may cause microbial inactivation. For example, a minimum irradiance of 0.02 milliwatts per square centimeter (mW/cm²) may cause microbial inactivation on a surface over time, e.g., several weeks. In some examples, an irradiance of 0.05 mW/cm² may inactivate microorganisms on a surface, but higher values such as 0.1 mW/cm², 0.5 mW/cm², 1 mW/cm², or 2 mW/cm² may be used for quicker microorganism inactivation. In some examples, even higher irradiances may be used over shorter periods of time, e.g., 3 to 10 mW/cm². In some examples where the light emitted is very close to the surface intended to be disinfected, e.g., 0.5 mm to 50 mm, even higher irradiances may be achieved, e.g., 10 to 50 mW/cm². Example light emitters disclosed herein may be configured to produce light with such irradiances at a given surface.

In some examples, light for microbial inactivation may include radiometric energy sufficient to inactive at least one bacterial population, or in some examples, a plurality of bacterial populations. One or more light emitters may have some minimum amount of radiometric energy (e.g., 10 mW, 20 mW, 100 mW, 1000 mW, or 3000 mW for one light emitter) measured from 380-420 nm light.

Dosage (measured in Joules/cm²) may be another metric for determining an appropriate irradiance for microbial inactivation over a period of time. Table 1 below shows example correlations between irradiance in mW/cm² and Joules/cm² based on different exposure times. These values are examples and many others may be possible.

TABLE 1

| Irradiance (mW/cm²) | Exposure Time (hours) | Dosage (Joules/cm²) |
|---|---|---|
| 0.02 | 1 | 0.072 |
| 0.02 | 24 | 1.728 |
| 0.02 | 250 | 18 |
| 0.02 | 500 | 36 |
| 0.02 | 1000 | 72 |
| 0.05 | 1 | 0.18 |
| 0.05 | 24 | 4.32 |
| 0.05 | 250 | 45 |
| 0.05 | 500 | 90 |
| 0.05 | 1000 | 180 |
| 0.1 | 1 | 0.36 |
| 0.1 | 24 | 8.64 |
| 0.1 | 250 | 90 |
| 0.1 | 500 | 180 |
| 0.1 | 1000 | 360 |
| 0.5 | 1 | 1.8 |
| 0.5 | 24 | 43.2 |
| 0.5 | 250 | 450 |
| 0.5 | 500 | 900 |
| 0.5 | 1000 | 1800 |
| 1 | 1 | 3.6 |
| 1 | 24 | 86.4 |
| 1 | 250 | 900 |
| 1 | 500 | 1800 |
| 1 | 1000 | 3600 |

Table 2 shows examples of dosages for inactivating example bacterial species using 405 nm light. Example dosages and other calculations shown herein may be examples from laboratory settings, and may not represent example dosages or calculations in other situations. For example, real world applications may require dosages or other calculations performed herein that may differ from example laboratory data. Inactivation may be measured by Log₁₀ reduction. Other dosages of 405 nm light may be used with other bacteria not listed below.

TABLE 2

| | Recommended Dose (J/cm²) for 1-Log |
|---|---|
| Organism | Reduction in Bacteria |
| Staphylococcus aureus | 20 |
| MRSA | 20 |
| Pseudomonas aeruginosa | 45 |
| Escherichia coli | 80 |
| Enterococcus faecalis | 90 |

Equation 2 may be used in order to determine irradiance, dosage, or time using one or more data points from Table 1 and Table 2:

$$\frac{Irradiance ( \frac{mW}{{cm}^{2}} )}{1000} * Time ( s ) = Dosage ( \frac{J}{{cm}^{2}} ) \qquad Equation 2$$

Irradiance may be determined based on dosage and time. For example, if a dosage of 30 Joules/cm² is desired and the object to be disinfected is going to be exposed to light overnight for 8 hours, the irradiance may be approximately 1 mW/cm². If a dosage of 50 Joules/cm² is desired and the object to be disinfected is going to be exposed to light for 48 hours, a smaller irradiance of approximately 0.3 mW/cm² may be sufficient.

A period of time to power the example light emitting subcomponents disclosed herein may be determined based on irradiance and dosage. For example, a device may be configured to emit an irradiance of disinfecting energy (e.g., 0.05 mW/cm²), and a target bacterium may require a dosage of 20 Joules/cm² to kill the target bacteria. Disinfecting light at 0.05 mW/cm² may have a minimum exposure time of approximately 4.6 days to achieve the dosage of 20 Joules/cm². Dosage values may be determined by a target reduction in bacteria. Once the bacteria count is reduced to a desired amount, disinfecting light may be continuously applied to keep the bacteria counts down.

Various colors of light may be utilized with a percentage (e.g., 75%) of their spectral power distribution within the wavelength range of 380-420 nm. In some examples, colors of light may be utilized with a percentage of 30% to 100% spectral power distribution within the wavelength range of 380-420 nm. In other examples, colors such as, for example, blue, green, and red, may be used with a minimum percentage of spectral energy (e.g., 20%) within the range of 380-420 nm, which may provide disinfecting energy.

A proportion of spectral energy may be determined by an amount of spectral energy within a specified wavelength range, e.g., 380-420 nm, divided by the total amount of spectral energy. Proportions of spectral energy may be presented as a percentage of the total amount of energy

In some examples, light provided in the wavelength range of 380-420 nm may not be adequate for general illumination purposes due to the violet color and low color quality characteristics. In some examples, this light may be used in spaces unoccupied by humans or spaces where additional white lighting for general illumination is already used. In some examples where a light emitting subcomponent is integrated into a device, e.g., humidifier, general white light illumination may not be required.

In some examples, continuous disinfection may be employed. For example, a surface intended to be disinfected may be continuously illuminated (e.g., illuminated 19 hours per day or multiple weeks at a time). A surface may be illuminated for a first percentage of time (e.g., 80% of the time) and not illuminated for a second percentage of time (e.g., 20% of the time). For example, a surface or object may not be illuminated when the object or surface is being interacted with, e.g., when using a cutting board. In some examples, intermittent disinfection may be employed, such as, for example, a surface may only be illuminated and disinfected at night, e.g., 8 hours per day.

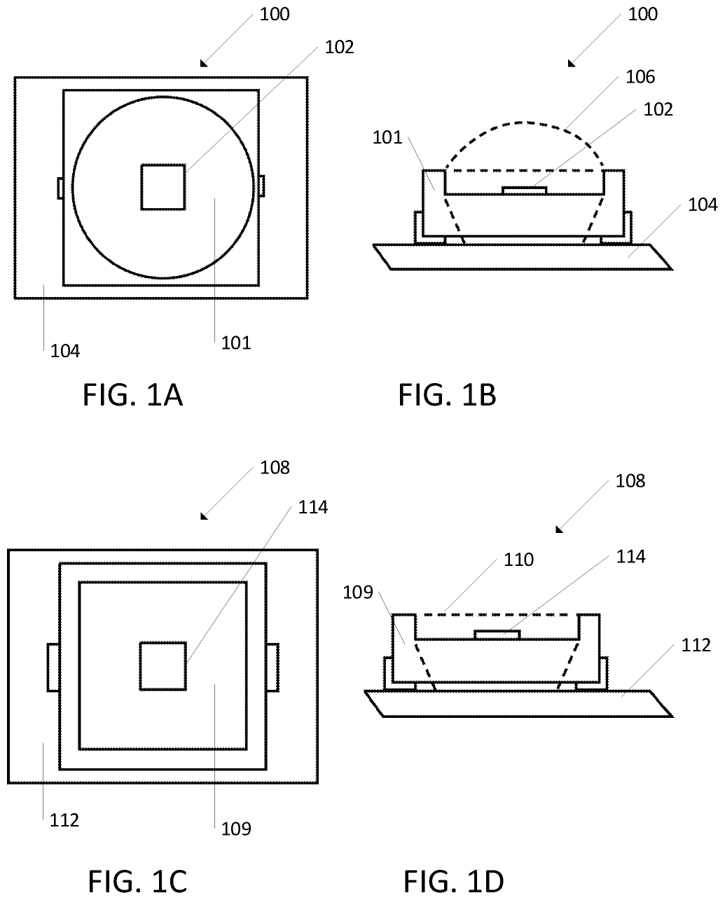

In examples disclosed herein, a light emitting subcomponent 100 may comprise a light emitter 101 as shown in FIGS. 1A-1B. The light emitter 101 may comprise a lighting element 102, e.g., an LED die. The light emitting subcomponent 100 may further comprise a substrate 104 and a circular light directing material 106, e.g., a lens. The light directing material 106 may cover the lighting element 102, such that light provided by the lighting element 102 may pass through the light directing material 106. In some examples, the light directing material 106 may be in a direct path of light emitted from the lighting element 102 without entirely covering the lighting element 102. In some examples, the light directing material 106 may comprise a domed shape over the lighting element 102 as shown in FIG. 1B.

In some examples, the lighting element 102 may comprise any device capable of emitting light, such as, for example, semiconductor die, LEDs, LEDs with light-converting layer(s), laser, electroluminescent wires, electroluminescent sheets, flexible LEDs, and OLEDs.

FIGS. 1C-1D illustrate a light emitting subcomponent 108 with a rectangular light directing material 110. The light emitting subcomponent 108 may comprise light emitter 109 and a substrate 112. The light emitter 109 may comprise a lighting element 114. The light directing material 110 may cover the lighting element 114, such that light provided by the lighting clement 114 may pass through the light directing material 110. In some examples, the light directing material 110 may be in a direct path of light emitted from the lighting element 114 without entirely covering the lighting element 114. The light directing material 110 may comprise a flat orientation perpendicular to light emitted by the lighting element 114 as is shown in FIG. 1D.

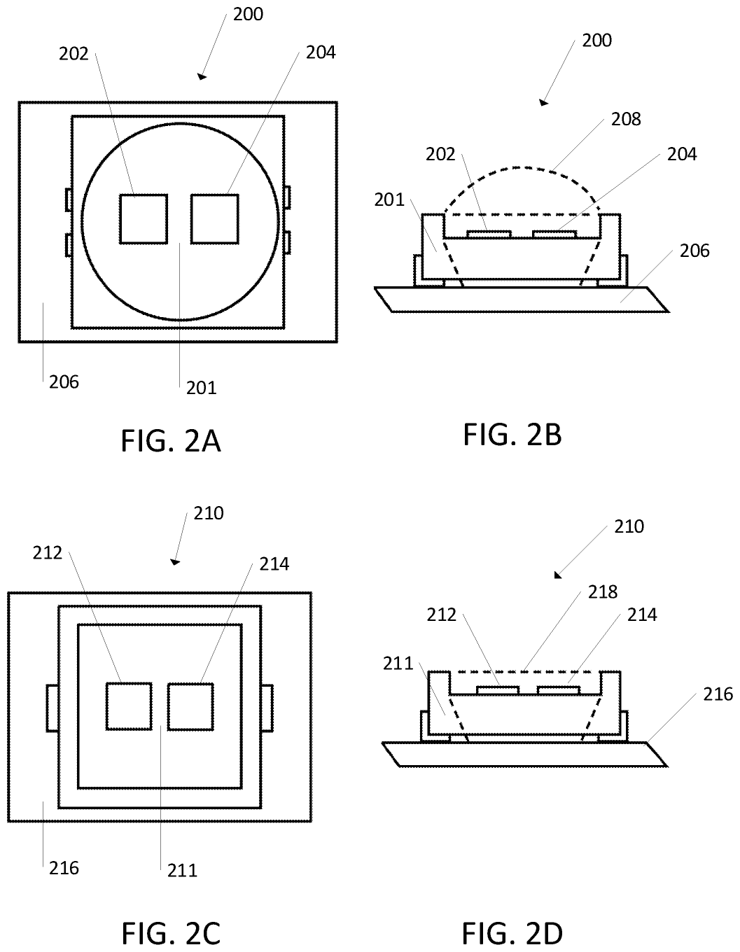

FIGS. 2A-2B show a light emitting subcomponent 200 that may comprise a light emitter 201. The light emitter 201 may comprise a first lighting element 202 and a second lighting element 204. In some examples, the first lighting element 202 and the second lighting element 204 may be the same and emit substantially similar light. In other examples, the first lighting element 202 and the second lighting element 204 may be different, e.g., emit light of different spectral power distributions. The light emitting subcomponent 200 may comprise a substrate 206. The light emitter 201 may comprise a circular light directing material 208. The light directing material 208 may cover the first lighting element 202 and the second lighting element 204 such that light provided by first lighting element 202 and the second lighting element 204 may pass through the light directing material 208. In some examples, the light directing material 208 may be in a direct path of light emitted from the first lighting element 202 and the second lighting element 204 without entirely covering the first lighting element 202 and the second lighting element 204. In some examples, the light directing material 208 may be in a direct path of light emitted from or entirely cover only one of the first lighting element 202 or the second lighting element 204. In some examples, the light directing material 208 may comprise a domed shape over the first lighting element 202 and the second lighting element 204 as shown in FIG. 2B.

FIGS. 2C-2D show a light emitting subcomponent 210 that may comprise a light emitter 211. The light emitter 211 may comprise a first lighting element 212 and a second lighting clement 214. In some examples, the first lighting clement 212 and the second lighting clement 214 may be the same and emit substantially similar light. In other examples, the first lighting element 212 and the second lighting element 214 may be different, e.g., emit light of different spectral power distributions. The light emitting subcomponent 210 may comprise a substrate 216 and a square light directing material 218. The light directing material 218 may cover the first lighting element 212 and the second lighting element 214 such that light provided by first lighting element 212 and the second lighting element 214 may pass through the light directing material 218. In some examples, the light directing material 218 may be in a direct path of light emitted from the first lighting element 212 and the second lighting element 214 without entirely covering first lighting element 212 and the second lighting element 214. In some examples, the light directing material 218 may be in a direct path of light emitted from or entirely cover only one of the first lighting element 212 or the second lighting element 214. In some examples, the light directing material 218 may comprise a domed shape over the first lighting element 212 and the second lighting element 214 as shown in FIG. 2D.

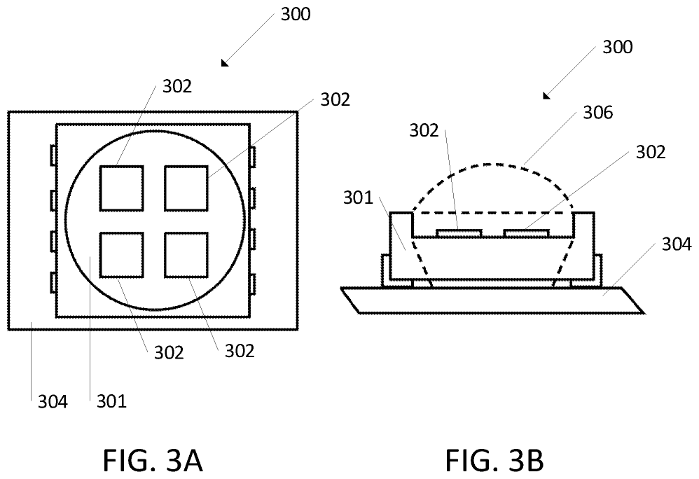

FIGS. 3A-3B show a light emitting subcomponent 300 that may comprise a light emitter 301. The light emitter 301 may comprise four lighting elements 302. In some examples, each of the four lighting elements 302 may be the same and emit substantially similar light. In other examples, the four lighting elements 302 may be different, e.g., emit light of different spectral power distributions. In other examples, any other combination of similar or dissimilar lighting elements 302 may be used. The light emitting subcomponent 300 may comprise a substrate 304 and a circular light directing material 306. The light directing material 306 may cover the lighting elements 302, such that light provided by the lighting elements 302 may pass through the light directing material 306. In some examples, the light directing material 306 may be in a direct path of light emitted from the lighting elements 302 without entirely covering the lighting elements 302. In some examples, the light directing material 306 may be in a direct path of light emitted from or entirely cover only number of the lighting elements 302. In some examples, the light directing material 306 may comprise a domed shape over the lighting elements 302 as shown in FIG. 3B.

In some examples, the light directing material 306 may comprise light converting materials such as, for example, filters, diffusers, and/or optics that may be used to provide optical filtering (e.g., high pass, bandpass, or low pass filter functionality). For example, if light within the range of 400-420 nm is desired, an optical filtering element may cut off wavelengths above, below, and/or outside a desired range. Optical filtering may reduce or block any potentially harmful wavelengths, e.g., below 380 nm. Optical filtering may reduce the risk of UV exposure or material degradation due to UV wavelengths. In some examples, a light converting material, filter, diffuser, and/or optical filtering elements that may be used to provide optical filtering may be external to the light emitting subcomponent 300, and in some examples, may cover the light emitter 301 or the entire light emitting subcomponent 300.

Material choice for lenses, diffusers, reflectors, optics, protective shields, and other transmissive components of a light fixture or light bulb may contribute to the overall efficiency of the light emitting subcomponents 100, 108, 200, 210, 300. The choice of material(s) may affect transmission and/or reflection efficiencies. Reflectors and/or transmissible components for general illumination sources (e.g., incandescent bulbs, fluorescent bulbs, and RGB/blue pump phosphor converted LEDS) may include, for example, plastics, glasses, coatings, and other materials. Incandescent bulbs, fluorescent bulbs, and RGB/blue pump phosphor converted LEDS may rely very little on near-UV wavelengths to provide illumination. Additionally, many of these reflectors or transmissive components may exhibit a sharp drop off in reflectance or transmission efficiency (e.g., close to zero in some examples) for wavelengths of light in the near-UV range, e.g., the wavelength is approximately 380 to 420 nm. Light fixtures or light bulbs made of improper reflective or transmissive materials may cause light in the 380-420 nm range to show a decrease in spectral energy. Reflective and transmissive materials may be selected based on the output of light from a light source. In some examples, a diffuser may allow at least 50% total transmission in the 380-420 nm range. In some examples, a diffuser may allow at least 75% total transmission in the 380-420 nm range.

In some examples, the material for a reflector or diffuser may be selected such that it may transmit as much 380-420 nm light as possible. In some examples, it may be desirable to select materials (e.g., plastics, resins, thermoplastic resins, or polymers) that may not substantially absorb light in the 380-420 nm range.

In some examples, the lighting element 102, 114, 202, 204, 212, 214, 302 may have a small full width at half maximum (FWHM) emission spectrum (e.g., 12.5 nm, 13.5 nm, 14.7 nm, 16 nm, 20 nm) in order to concentrate energy surrounding a peak of approximately 405 nm. Concentrating energy to a target range where it provides disinfection minimizes waste of non-useful energy outside of the target range. Concentrating energy to a FWHM emission spectrum may increase safety by reducing the intensity of wavelengths that may bleed into the UV range. In some examples, the FWHM may be less than 20 nm, between 12 and 20 nm, and/or 13.5 nm. In some examples, the light directing material 106, 110, 208, 218, 306 may provide the desired small FWHM emission spectrum. In some examples, the light directing material 106, 110, 208, 218, 306 may provide the desired small FWHM emission spectrum by, for example, providing filtering or light conversion.

In some examples, the light directing material 106, 110, 208, 218, 306 may comprise a phosphor, an optical brightener, a combination of phosphors, a combination of optical brighteners, or a combination of phosphor(s) and optical brightener(s). In some examples, the light directing material 106, 110, 208, 218, 306 may comprise quantum dots, a phosphorescent material, a fluorophore, a fluorescent dye, a conductive polymer, or a combination of any one or more types of light-converting materials. In some examples, the light directing material 106, 110, 208, 218, 306 may comprise an activator (e.g., a light-converting element) and a host (e.g., a non-light-converting element). A phosphor or other light-converting material may be deposited directly on a light emitting subcomponent, as disclosed herein, or may be remote or further removed from the lighting element 102, 114, 202, 204, 212, 214, 302. Light-converting materials may be deposited, for example, as conformal coatings, doped encapsulants, binder materials, or remote phosphors.

In some examples, it may be desirable to dissipate heat generated by lighting elements or other components of a light emitter as disclosed herein. A decreased operating temperature may increase reliability and lifetime of a device. Heat may affect the peak wavelength and spectrum emitted by the LEDs. For example, as temperatures rise, peak wavelengths may shift to longer wavelengths and/or the entire spectrum may be moved away from UV light and towards visible light. Similarly, as temperatures decrease, peak wavelengths may shift to shorter wavelengths and/or and the entire spectrum may be moved away from visible light and towards UV light. Therefore, it may be desirable to constrain the temperature to a certain range in order to maintain a desired peak wavelength or spectrum within some tolerance. In some examples, the light emitter may be coupled to a heatsink (not shown). The heatsink may be made out of plastics, ceramics, or metals including, for example, aluminum, steel, or copper. The heatsink may also be made out of a plastic or ceramic material. In some examples the heatsink may be permanently coupled to a light emitter, or otherwise considered a part of the assembly that makes up the light emitter or light emitting subcomponent.

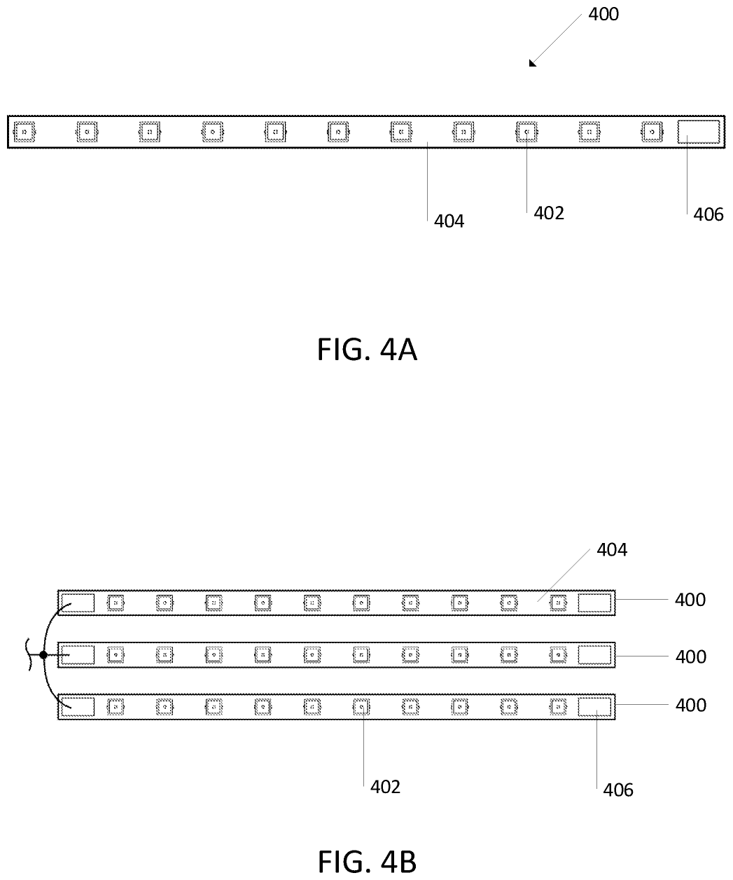

In some examples, a substrate 104, 112, 206, 216, 304, 404 of a light emitting subcomponent 400 with light emitters 101, 109, 201, 211, 301 may comprise a circuit board or printed circuit board as shown in FIG. 4A. In some examples, the circuit board of the light emitting subcomponent 400 may be populated with light emitters 402. In some examples, the circuit board may contain or be connected to control circuitry 406 that may control the light emitted from the light emitters 402. In some examples, the control circuitry 406 may determine the number of light emitters 402 to be powered. The control circuitry 406 may control the light emitters 402 and determine which light emitters 402 to turn on based on a number of factors such as, for example, distance between the light emitting subcomponent 400 and a surface to be disinfected, time, a threshold irradiance necessary to initiate inactivation of a microorganism to be inactivated, and the irradiance provided by a single light emitter 402. The control circuitry 406 may also control, for example, the amount of time the light emitters 402 are on or off, and may comprise occupancy sensors, e.g., motion sensors. The control circuitry 406 may be configured to adjust the light emitting subcomponent 400 based on the occupancy sensors, e.g., adjusts the concentration of spectral energy at specific wavelengths.

The light emitters 402 and control circuitry 406 may be configured to connect to a power source (not shown). In some examples, the light emitters 402, light emitting subcomponents 400, light fixtures, or devices disclosed herein may be powered through power outlets, electrical power supplies, batteries or rechargeable batteries, and/or wireless or inductive charging. In some examples that comprise rechargeable batteries, the rechargeable batteries may be recharged by, for example, AC power or solar panels. In some examples, AC power and an AC to DC converter, e.g., an LED driver or power supply, may be utilized. In some examples, direct DC power may be utilized when available. In some examples, wireless or inductive charging may charge or power the light fixture or device.

The substrate 404 of a light emitting subcomponent 400 may vary in material, shape, size, thickness, flexibility, and otherwise be conformed to specific applications. Base material of the substrate 404 may comprise a variety of materials such as, for example, aluminum, FR-4 (glass-reinforced epoxy laminate material), Teflon, polyimide, or copper.

FIGS. 4A-6 show a number of different light emitting subcomponent shapes, such as, for example, straight, circular, or rectangular. An array of light emitting subcomponents may be formed from multiple light emitting subcomponents 400. In some example, an array of light emitting subcomponents may comprise multiple connected light emitting subcomponents 400 as shown in FIG. 4B. The control circuitry 406 may vary the shape of the array that is providing illumination based on determining which light emitters 402 to power, e.g., the control circuitry 406 may power a portion of the light emitters 402 to illuminate a smaller area than may be covered by illuminating the entire array of light emitting subcomponents 400.

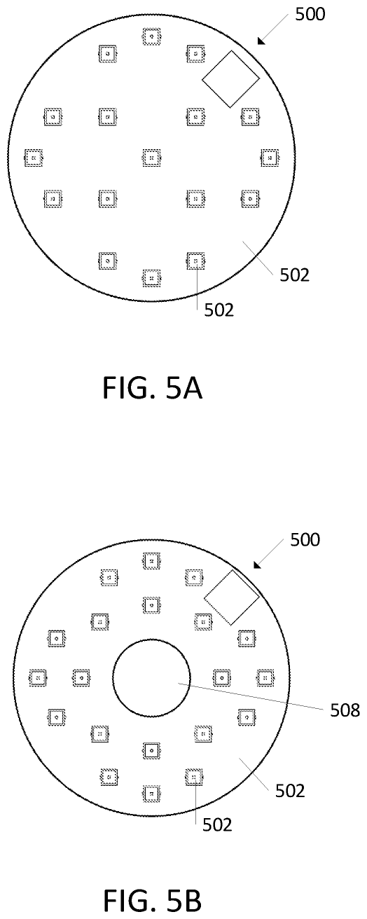

In some examples, a light emitting subcomponent 500 may form a circle as shown in FIGS. 5A-5B. FIG. 5A shows a light emitting subcomponent 500 with a circular substrate 504 and light emitters 502 arranged circularly thereon. FIG. 5B shows a similar light emitting subcomponent 500 where the circular substrate 504 has a hole 506 within it.

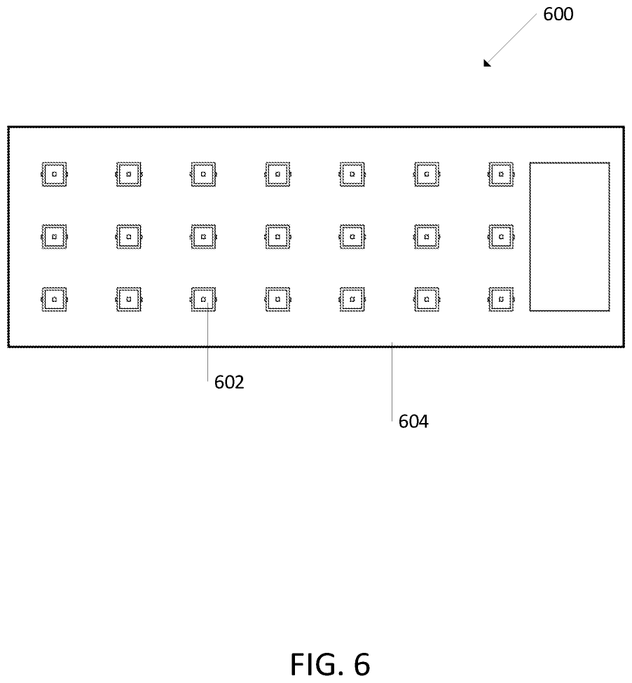

In some examples, a light emitting subcomponent 600 may form a rectangle as shown in FIG. 6. FIG. 6 shows a light emitting subcomponent 600 with a rectangular substrate 604 and light emitters 602 arranged thereon.

In some examples, a light emitter or a light emitting subcomponent may comprise a conformal coating. The conformal coating may comprise a polymeric film contoured to the light emitting subcomponent. The conformal coatings may provide ingress protection from, for example, condensation or other liquids.

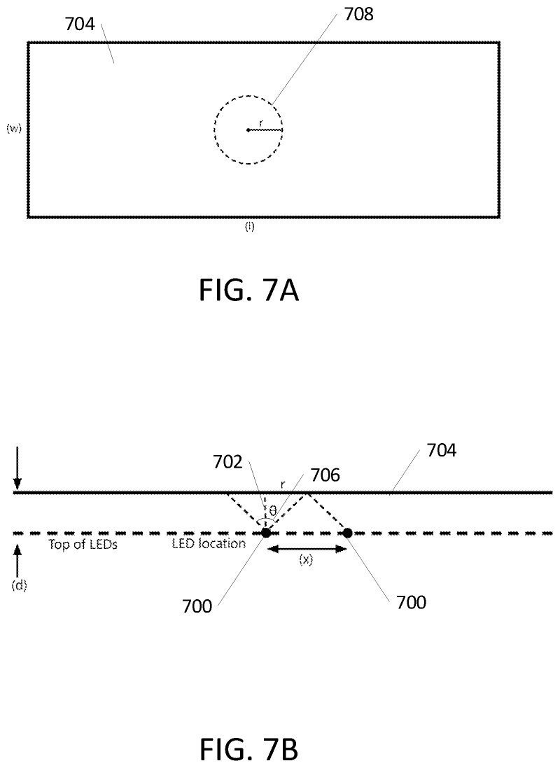

In some examples, a surface to be disinfected may be in close proximity to a light emitting subcomponent. In such examples, a light emitting subcomponent or an array of light emitting subcomponents may require more light emitters than would otherwise be necessary for disinfection. The area illuminated by a single light emitter may be limited by a beam angle of the light emitter. The same light emitter may illuminate a larger surface area of the surface to be disinfected if the light emitter is moved further away. Therefore, a light emitting subcomponent may need an increased number of light emitters to cover the entire surface area of the surface to be disinfected with disinfecting light, as compared to a light emitting subcomponents at a further distance. FIG. 7A illustrates angles of light emitted from light emitters disclosed herein. Light emitters 700 may be spaced a distance 702 from the surface 704 to be disinfected. The light emitters 700 may emit a light that spreads outwardly toward the surface 704 at a beam angle 706. The beam angle 706 may comprise half of an angle of light emitted from the light emitter 700, in degrees, where the intensity of light is at least 50% of light emitter's 700 maximum emission intensity. In some examples, the light emitter 700 may comprise LEDs and the beam angle 706 may be 130 degrees, e.g., the angle of light emitted from the light emitter where the intensity of light is at least 50% of the maximum emission intensity is 130 degrees. In some examples where light from the light emitter 700 does not possess rational symmetry, the beam angle 702 may be given for two planes at 90 degrees to each other.

A total surface area illuminated by one light emitter 708, as shown in FIG. 7B, may be determined by the beam angle 706 and the distance 702 from the light emitter 700 to the surface 704 intended to be disinfected. A light emitter 700 with a larger beam angle 706 may provide a larger total surface area illuminated by one light emitter 708. An increased distance 702 between the light emitter 700 and the surface 704 may also increase the total surface area illuminated by one light emitter 708. The total number of light emitters 700 that may be needed to disinfect the entire surface 704 to be disinfected may be based on the total surface area illuminated by one light emitter 708. As the distance 702 from the surface 704 intended to be disinfected to the light emitter 700 decreases, the number of light emitters 700 that may be needed to disinfect the surface may increase.

In some examples, the surface 704 intended to be disinfected may be a protective layer over the light emitters 700. In other examples, the surface 704 to be disinfected may be an object that may be placed on top of a protective surface over the light emitters 700, e.g., a cell phone. In some examples, the total surface area illuminated by one light emitter 708 may be substantially the same as a surface area the surface 704 to be disinfected, such as, for example, when the light emitter 700 is close (e.g., 2 cm or less) to the surface 704 to be disinfected. In some examples the total surface area illuminated by one light emitter 708 may not be the same as the surface area the surface 704 to be disinfected.

In one example, the total surface area illuminated by a light emitting subcomponent may be 15 cm×15 cm and the surface area the surface 704 to be disinfected may be substantially the same size. If the distance 702 from the light emitting subcomponent to the surface 704 is 1.5 cm, and the light emitters 700 have a beam angle of 130 degrees, the total surface area illuminated by one light emitter 708 may be 32.5 cm², and the light emitting subcomponent may need 7 light emitters 700 to cover the entire surface 704. In another example, the total surface area illuminated by a light emitting subcomponent may be 100 cm×100 cm and the surface area the surface 704 to be disinfected may be substantially the same size. If the distance 702 from the light emitting subcomponent to the surface 704 is 1 cm, and the light emitters 700 have a beam angle of 130 degrees, the light emitting subcomponent may need 693 light emitters 700. In another example, the total surface area illuminated by a light emitting subcomponent may be 100 cm×100 cm and the surface area the surface 704 to be disinfected may be substantially the same size. If the distance 702 from the light emitting subcomponent to the surface 704 is 4 cm, and the light emitters 700 have a beam angle of 130 degrees, the total surface area illuminated by one light emitter 708 may be 231.2 cm², and light emitting subcomponent may need 44 light emitters 700.

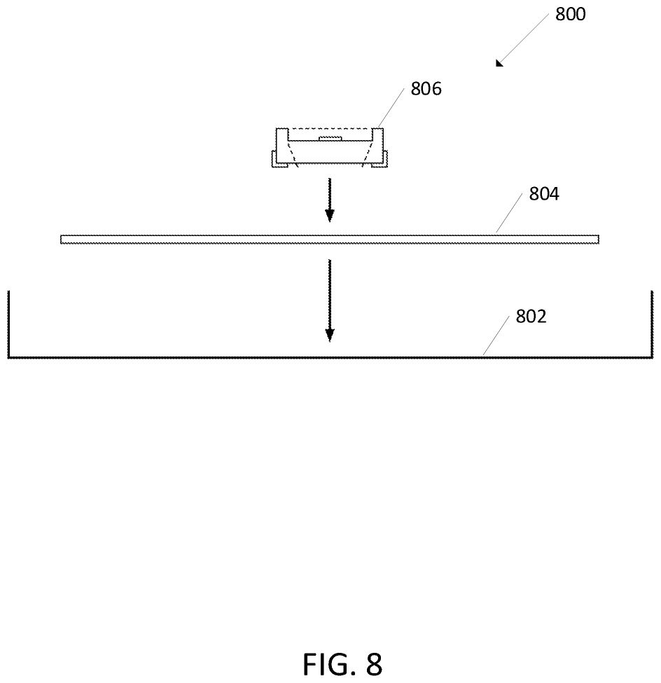

In some examples, a light emitting subcomponent may be configured to be integrated into an assembly of a fixture or device. FIG. 8 shows a lighting device 800. The lighting device 800 may comprise a light housing 802. A light emitting subcomponent may be attached or otherwise mounted to or within the light housing 802. The light emitting subcomponent may comprise a substrate 804 with a light emitter 806 forming the light emitting subcomponent. The fixture or device that may comprise the light housing 802 may be, for example, a humidifier (not shown).

By way of example, various light emitters and light emitting subcomponents and features are discussed below. Such features may be separated, combined, rearranged, and/or used together. In some examples, a light emitter may comprise a surface mount LED device, which may include an LED and a light-conversion material. The surface mount LED device may, in some examples, be mounted onto a printed circuit board (“PCB”) or otherwise configured to transfer power to the light-emitting device and to the LED. The LED may be coupled to the PCB through bond wires or leads which enable an electrical connection from the LED to the outside of the device. In some examples, the surface mount LED device may have a lens, encapsulants, or other protective cover.

In some examples, the light emitter may comprise a chip scale package (CSP) or a flip chip CSP, which may package the light emitters without using a traditional ceramic/plastic package and/or bond wires, and may enable a substrate to be attached directly to the PCB.

In some examples, there may be more than one light emitter and the light emitters may be arranged in an approximately evenly spaced array.

In some examples, a light emitting subcomponent may comprise an array of LEDs contained by a light-converting material that may be contained by an encapsulant and supported on a substrate.

In some examples, a light fixture or device may be integrated with one or more light emitting subcomponents. The light fixture or device may comprise additional materials and components, e.g., LED drivers, housings, plastic diffusers, endcaps, wiring harnesses, control circuitry, hardware, and/or means for mounting the light emitting subcomponent.

In some examples, the light emitting subcomponent may be a circuit board.

In some examples, the light emitting subcomponent may be made of aluminum.

In some examples, the light emitting subcomponent may be made of FR-4.

In some examples, the light emitting subcomponent may be populated with one or more light emitters.

In some examples, the one or more light emitters may be one or more light emitting diodes (LEDs).

In some examples, the one or more light emitters may emit light in a range of 380-420 nm.

In some examples, the one or more light emitters may emit a light with a peak wavelength of approximately 405 nm.

In some examples, the light emitted by the one or more light emitters may be substantially the same.

In some examples, the light emitted by the one or more light emitters may have a full width half max of no more than 20 nm.

In some examples, the light emitting subcomponent may be flexible.

In some examples, the light emitting subcomponent may be rigid.

In some examples, the one or more light emitters may be configured to provide an irradiance on a target surface of at least 0.1 mW/cm².

In some examples, the one or more light emitters may be configured to provide an irradiance on a target surface of between 0.1 and 10 mW/cm².

In some examples, the light emitting subcomponent may be configured to inactivate microorganisms on a surface.

In some examples, the light emitting subcomponent may comprise or otherwise be connected to driving circuitry that may control the light output.

In some examples, the size and flexibility of the light emitting subcomponent may be customized for various applications.

In some examples, the light emitting subcomponent may comprise an adhesive back.

In some examples, the light emitting subcomponent may comprise components other than light emitters.

In some examples, only one side of the light emitting subcomponent may be populated with light emitters and/or components.

In some examples, both sides of the light emitting subcomponent may be populated with light emitters and/or components.

In some examples, the light emitting subcomponent may be connected to an LED driver that controls the voltage and current provided the light emitting subcomponent.

In some examples, the control circuitry may be able to control the output of the light including the brightness and/or duration of illumination.

In some examples the light emitting subcomponent may be conformal coated for protection of the one or more light emitters.

In some examples, a light emitting device may comprise a substrate and one or more light emitters disposed on the substrate. The light emitters may be configured to inactivate microorganisms on a surface a distance away from the substrate by emitting a light. The light may comprise a proportion of a spectral energy of the light, measured in a 380 nanometers (nm) to 420 nm wavelength range, greater than 50%. The light may comprise a full width half max (FWHM) emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface.

In some examples, the irradiance at the surface sufficient to initiate inactivation of microorganisms on the surface may comprise at least 0.02 milliwatts per square centimeter (mW/cm²).

In some examples, the light emitting device may comprise a sensor configured to detect occupancy of an area comprising the surface. The light emitting device may comprise a controller configured to adjust, based on the sensor detecting occupancy of the area, the proportion of the spectral energy, measured in the 380 nm to 420 nm wavelength range, of the light between 0% and 100%.

In some examples where the light is a first light, the light emitting device may comprise a conversion material arranged in a direct path of the first light and configured to generate, based on the first light, a second light comprising a wavelength outside the 380 nm to 420 nm wavelength range.

In some examples, the substrate of a light emitting device may comprise one or more of aluminum, glass-reinforced epoxy laminate, Teflon, polyimide, or copper.

In some examples, the light emitters may comprise one or more light emitting diodes (LEDs).

In some examples, one or more light emitters may be configured as an array of light emitters on the substrate.

In some examples, a number of the one or more light emitters may be determined based on the distance that the surface is away from the substrate, based on the size of the surface, based on a microorganism to be inactivated, or based on a beam angle of at least one of the one or more light emitters.

In some examples, one or more light emitters may be arranged based on the distance that the surface is away from the substrate, based on the size of the surface, based on a microorganism to be inactivated, or based on a beam angle of at least one of the one or more light emitters.

In some examples, a radiometric energy of the light emitted by the one or more light emitters may be based on the distance that the surface is away from the substrate, based on the size of the surface, based on a microorganism to be inactivated, or based on a beam angle of at least one of the one or more light emitters.

In some examples, the FWHM emission spectrum of the light from the light emitting device may comprise an FWHM emission spectrum corresponding to light measured in the 380 nm to 420 nm wavelength range.

In some examples, a method may comprise emitting, via one or more light emitters disposed on a substrate and to inactivate microorganisms on a surface a distance away from the substrate, a light. The light may comprise a proportion of a spectral energy of the light, measured in a 380 nm to 420 nm wavelength range, greater than 50%. The light may comprise a FWHM emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface. The light may cause, based on emission of the light, inactivation of the microorganisms on the surface.

In some examples of the method, the irradiance at the surface sufficient to initiate inactivation of microorganisms on the surface may comprise at least 0.02 milliwatts per square centimeter (mW/cm²).

In some examples, the method may comprise detecting, via a sensor, occupancy of an area comprising the surface. The method may comprise adjusting, via a controller in communication with the sensor and the one or more light emitters and based on the detecting occupancy of the area, the proportion of spectral energy, measured in the 380 nm to 420 nm wavelength range, of light between 0% and 100%.

Some examples of the method, where the light is a first light, may comprise causing conversion of the first light, via a conversion material arranged in a direct path of the first light, of the first light to a second light comprising a wavelength outside the 380 nm to 420 nm wavelength range.

In some examples, the method may comprise configuring the one or more light emitters as an array of light emitters on the substrate.

Some examples of the method may comprise determining the distance between the light emitters and the surface and determining a size of the surface. The method may further comprise determining, based on the determined distance between the light emitters and the surface, based on the size of the surface, based on a microorganism to be inactivated, or based on a beam angle of at least one of the one or more light emitters, a number of light emitters to inactivate the microorganism. The method may comprise powering the determined number of light emitters for a period of time.

In some examples, the one or more light emitters may be arranged based on the distance that the surface is away from the substrate, based on a size of the surface, based on a microorganism to be inactivated, or based on a beam angle of at least one of the one or more light emitters.

In some examples, a radiometric energy of the light emitted by the one or more light emitters may be based on the distance that the surface is away from the substrate, based on a size of the surface, based on a microorganism to be inactivated, or based on a beam angle of at least one of the one or more light emitters.

In some examples, a light emitting device may comprise an array of light emitting subcomponents. The light emitting subcomponents may be configured to inactivate microorganisms on a surface a distance away from the light emitting subcomponents by emitting a light. The light may comprise a proportion of a spectral energy of the light, measured in a 380 nanometers (nm) to 420 nm wavelength range, greater than 50%. The light may comprise a FWHM emission spectrum of less than 20 nm and centered at a wavelength of approximately 405 nm to concentrate the spectral energy of the light and minimize energy associated with wavelengths that bleed into an ultraviolet wavelength range. The light may comprise an irradiance at the surface sufficient to initiate inactivation of microorganisms on the surface. The light emitting device may comprise a controller disposed on the substrate and configured to adjust output of the array of light emitting subcomponents.

In some examples, the controller may be configured to adjust, based on a time that the light emitted by the array of light emitting subcomponents has been emitted, a radiometric energy of the light emitted by the array of light emitting subcomponents.

In some examples, the controller of the light emitting device may be configured to adjust the proportion of the spectral energy, measured in a 380 nm to 420 nm wavelength range, between 0% and 100%.

In some examples, the controller may be configured to determine the distance between the array of light emitting subcomponents and the surface and determine a size of the surface. The controller may be configured to determine, based on the determined distance between the array of light emitting subcomponents and the surface, based on the size of the surface, based on a microorganism to be inactivated, or based on a beam angle of at least one light emitting subcomponent, a number of light emitting subcomponents to inactivate the microorganism. The controller may be configured to power the determined number of light emitting subcomponents for a period of time.

In some examples, the light emitting subcomponents may be arranged based on the distance between array of light emitting subcomponents and the surface, based on a size of the surface, based on a microorganism to be inactivated, or based on a beam angle of at least one of the light emitting subcomponents.

In some examples, a radiometric energy of the light emitted by the light emitting subcomponents may be based on a distance between the array of light emitting subcomponents and the surface, based on a size of the surface, based on a microorganism to be inactivated, or based on a beam angle of at least one of the light emitting subcomponents.

Approximating language, as used herein throughout the specification and claims, may be applied to modify any quantitative representation that could permissibly vary without resulting in a change in the basic function to which it is related. Accordingly, a value modified by a term or terms, such as “about”, “approximately” and “substantially”, are not to be limited to the precise value specified. Values identified herein may be varied between +/− 10% of the stated value(s) and still function as described. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The above disclosure sets forth various examples. Modifications of which may be made as desired for different implementations. For example, steps and/or components may be subdivided, combined, rearranged, removed, and/or augmented; performed on a single device or a plurality of devices; performed in parallel, in series; or any combination thereof. Additional features may be added.

1. A light emitting device comprising:
a circuit board; and
at least one light emitter disposed on the circuit board and configured to emit a light to inactivate microorganisms and cause a target irradiance at a surface a distance away from the circuit board wherein the circuit board comprises an on-board controller, and wherein the light emitted by the one or more light emitters comprises a wavelength in the range of 380 nanometers (nm) to 420 nm.

2. The light emitting device of claim 1, wherein the circuit board is in a form of a circle with a hole in the middle.

3. The light emitting device of claim 1, wherein a proportion of spectral energy of the light, measured in a 380 nanometers (nm) to 420 nm wavelength range, is at least 50%.

4. The light emitting device of claim 1, wherein the at least one light emitter further comprises a full width half max (FWHM) emission spectrum of less than 20 nm and centered at a wavelength of approximately 405 nm to concentrate a spectral energy of the light and minimize energy associated with wavelengths that bleed into an ultraviolet wavelength range.

5. The light emitting device of claim 1, wherein the at least one light emitter is further configured to produce a radiometric power that satisfies a threshold, wherein the threshold is based on the target irradiance at the surface.

6. The light emitting device of claim 1, wherein the target irradiance is at least 0.50 milliwatt per square centimeter (mW/cm2) across at least a portion of the surface.

7. The light emitting device of claim 1, further comprising a second controller, wherein the second controller is further configured to adjust the radiometric power emitted by the at least one light emitter based on a time that the light emitted by the at least one light emitter has been emitted.

8. The light emitting device of claim 1, wherein the on-board controller further comprises a driving circuitry component that controls characteristics of the emitted light, wherein the characteristic may be a radiometric power or duration of illumination.

9. The light emitting device of claim 1, wherein the circuit board is a printed circuit board configured to transfer power to the at least one light emitter.

10. The light emitting device of claim 1, wherein the at least one light emitter further comprises a conformal coating.

11. The light emitting device of claim 1, further comprising an array of light emitters, wherein the array of light emitters are configured to produce a combined radiometric power that satisfies a threshold, wherein the threshold is based on a target irradiance at the surface.

12. The light emitting device of claim 11, wherein the array of light emitters are arranged on the circuit board such that boundaries of the light emitted from neighboring light emitters of the array of emitters intersect or overlap at the surface.

13. The light emitting device of claim 1, wherein the on-board controller is configured to cause the at least one light emitter to continuously emit the light at a radiometric power that satisfies a target threshold.

14. The light emitting device of claim 1, wherein the circuit board is further configured to be integrated into an assembly of a fixture or device.

15. The light emitting device of claim 1, wherein at least a portion of the light is normal to a portion of the surface.

16. The light emitting device of claim 1, further comprising at least two light emitters, wherein the radiometric power emitted by the at least two light emitters is the same for each light emitter.

17. A light emitting device comprising:
a circuit board in a form of a circle with a hole in the middle; and
a circular array of light emitters disposed on the circuit board and configured to emit a light to inactivate microorganisms and cause a target irradiance on a surface a distance away from the circuit board, wherein the circuit board comprises an on-board controller, and wherein the light emitted by the array of light emitters comprises a wavelength in the range of 380 nanometers (nm) to 420 nm.

18. The light emitting device of claim 17, further comprising a second circuit board, wherein the circuit board and the second circuit board are coupled to each other, and wherein the circuit board the second circuit board each comprise an array of light emitters configured to produce the target irradiance.

19. The light emitting device of claim 17, wherein the on-board controller is configured to cause the light emitters to continuously emit the light at a radiometric power that satisfies a target threshold.

20. The light emitting device of claim 17, wherein the circuit board is disposed behind a diffuser, and wherein the diffuser allows for at least 75% of the light within the range of 380-420 nm to transmit through.
